(19) United States
(12) Patent Application Publication
(10) Pub. No.: US 2022/0036982 A1
(43) Pub. Date: Feb. 3, 2022

(54) METHODS FOR USING TEMPORARY INFORMATIONAL MARKINGS

(71) Applicant: Cambridge Research & Development, Inc., Nashua, NH (US)

(72) Inventors: Kenneth D. Steinberg, Nashua, NH (US); Jason W. Clark, Milford, NH (US); David L. Hall, Hollis, NH (US)

(21) Appl. No.: 16/944,771

(22) Filed: Jul. 31, 2020

(51) Int. Cl. G16H 10/60; G16H 40/67; G16H 15/00; G06K 19/06; G06K 19/077; A61Q 1/02

(52) U.S. Cl. CPC G16H 10/60; G16H 40/67; A61Q 1/025; G06K 19/0614; G06K 19/07758; G16H 15/00

(57) ABSTRACT

Methods for transmitting data between parties or locations using temporary markings that are affixed to a human or animal body are disclosed, along with related devices. These markings, or tattoos, are intended to be temporary, where the lifespan of the marking is dictated by the marking material used and the material's interaction with the dermis of the wearer. The markings can be visible, invisible or a combination thereof, placed in predetermined or random locations. The markings can also be affixed in visible or hidden locations, depending on privacy concerns.

### CROSS-REFERENCE TO RELATED APPLICATION

The application claims priority to U.S. Provisional Patent Application No. 62/831,299, filed on Apr. 9, 2019, the contents of which are incorporated by reference herein.

### FIELD OF THE DISCLOSURE

The disclosure relates to methods of using temporary markings, like tattoos, on humans and/or animals to communicate information between parties.

### BACKGROUND

Throughout society there are instances where one party needs to convey information to another when either verbal communication or the intricacies of the data are problematic. In many cases, solutions such as labels, bands, tags and other high- and low-tech solutions are applied.

These solutions, whereby, a person is affixed with some sort of means to store and communicate information, are often problematic as they can get lost, get damaged, or require additional componentry to read.

An ongoing example of this is the manner in which the US Armed Forces communicate medical information between medics and other care givers. When a warfighter is injured in battle, a medic must not only ascertain the extent of the injury but also provide treatment that will stabilize the warfighter until they can be medically evacuated to a triage location. In early days, a medic would record the injuries and treatment on a simple piece of paper, called a Tactical Combat Casualty Care (TC3) card and attach the card to the warfighter prior to medivac. Upon arriving at the medical triage location, caregivers can read the card and determine what care has already been given. Unfortunately, these TC3 cards were regularly lost.

In present times, this information is captured on a computer tablet, but the communication problem remains. It is highly unlikely, due to communications jamming, that a medic will be able to transmit this medical information to the triage center before the injured warfighter arrives. This creates the same communication issues as the loss of a paper TC3 card. A loss of communication can also occur with any communication device affixed to a warfighter as they can get lost at any point along the continuum of care.

The attached record keeping devices also impair the ability of care givers along the continuum of care to add additional information about changes in a patient's status and/or other stabilization actions that may have been taken. If a medic in a medivac helicopter must administer additional care, this must also be recorded and shared with those at the triage center.

It is important not only to make sure communication is not interrupted by the loss of attached data recording solutions, but attention also needs to be given to cases where information changes over time or the information is too voluminous to fit into or on a wearable device.

A typical example of such a case would include the use of medical bracelets and the fact that medical information, such as medication taken, can change frequently. The American Association of Retired Persons (AARP) polled their 38 Million members with respect to their priority of concerns. One of the top concerns was a loss of medical information and pharmaceuticals during travel. As one would expect, it is troubling not to be able to communicate all of a person's medical information to a care giver when abroad. This lack of knowledge can lead to significant complications that can have effects on short- and long-term outcomes. To combat this potential problem, many seniors turn to medical bracelets and carrying a copy of their medical information with them. Unfortunately, if the patient is not conscious or the medical information is too large to engrave on a band, there can be a loss of critical information communication.

There are many more such use cases where information must be communicated, on a temporary (days, weeks, months etc.) basis, but the limitations and risks of using labels or wearables is problematic. It is to this problem space that the present disclosure is primarily directed.

### SUMMARY

The present disclosure, most fundamentally stated, involves the use of temporary markings, or tattoos, to communicate information between parties for a short period of time. Instead of relying upon wearable or attached information tags, the present disclosure describes tattoos, in the form of either custom or industry standard markings to encode and communicate information.

The disclosed temporary markings can take the form of readable text or can be encoded using any number of schemes such as bar codes, QR codes, linguistic encoding, symbol encoding, mathematical, and/or audio encoding. Imprinting the marking on the skin of the human or animal can be performed using printing, tattoo, makeup devices, or by hand. Reading the encoded information can be performed by use of the human eye, a mobile phone, computer, or customized device.

The markings, which can be made using temporary elements such as inks, makeup, or injectable particles, are intended only to be used for a short period of time as the marking material is eventually shed by the body of the human or animal.

The longevity of the marking is a factor of the disclosed marking methods and is thereby customizable by the user. The material used to create the temporary marking can be applied to the epidermis of the wearer or any skin layer depending on the process by which the marking material is shed or absorbed.

In some embodiments, the information contained in the temporary markings might be structured to support industry standards, such as bar codes or custom data, for example, Universal Resource Locators (URLs) and application-specific bit maps.

In specific embodiments, information encoded into a marking is invisible to the human eye and thus requires additional componentry to expose the marking. Marking material that is reactive to other chemicals or light sources can be used, in some embodiments. For example, Ultraviolet-sensitive chemicals that are only visible using ultraviolet light may be used, in some embodiments.

In other embodiments, more than one marking may be used to communicate additional information or changes in information. The additional information could be captured using markings in addition to those already applied or as modifications of the original markings or the erasure and replacement of the original markings.

In select embodiments, a marking might include the use of both encoded and human readable markings. This could be used, for example, to identify a person or animal but protect the contents of some pertinent information using non-readable encoding. In some such embodiments, it might also be feasible to include multiple types of encoding. For example, some embodiments may utilize readable, bar, and QR encoded information simultaneously.

In some embodiments, the marking(s) may be applied by a transfer method in which the marking material is deposited onto a secondary material which is then used to transfer the marking to the individual.

In select embodiments, the temporary period for the markings can be extended by reinforcing the original markings with new temporary material or even a permanent marking material.

In yet another embodiment, a combination of temporary and permanent markings can be used such that information could be exchanged and changed over time by modifying the original permanent marking. Such example methods could include changing existing markings by overwriting portions with neutral tones, alternate colors, and/or invisible inks.

The features and advantages described herein are not all-inclusive and, in particular, many additional features and advantages will be apparent to one of ordinary skill in the art in view of the drawings, specification, and claims. Moreover, it should be noted that the language used in the specification has been principally selected for readability and instructional purposes, and not to limit the scope of the inventive subject matter.

### BRIEF DESCRIPTION OF THE DRAWINGS

FIGS. 1A-1F illustrate currently available wearable devices used to communicate medical information. In particular, FIGS. 1A and 1C illustrate example medical necklaces, FIGS. 1B, 1D, and 1E illustrate example medical bracelets, and

FIG. 1F illustrates an example medical care card.

FIGS. 2A and 2B illustrate exemplary techniques and componentry that may be used to apply the presently disclosed temporary markings.

FIG. 3 shows example flow diagram illustrating an exemplary process flow for encoding and decoding a temporary marking, in accordance with some embodiments of the subject disclosure.

FIG. 4 illustrates an exemplary method of temporarily marking a patient with medical information, in accordance with various embodiments of the subject application.

FIG. 5 illustrates an exemplary method of reading a temporary marking on a patient, in accordance with various embodiments of the subject application.

FIG. 6 illustrates an exemplary method of transmitting medical data across a communication gap, in accordance with various embodiments of the subject application.

FIG. 7 illustrates exemplary types of injuries and related data that can be encoded into a temporary marking described herein.

### DETAILED DESCRIPTION

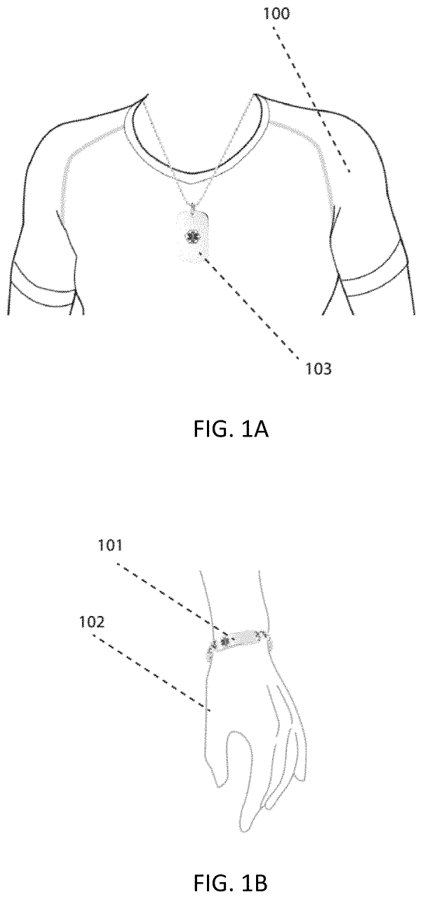

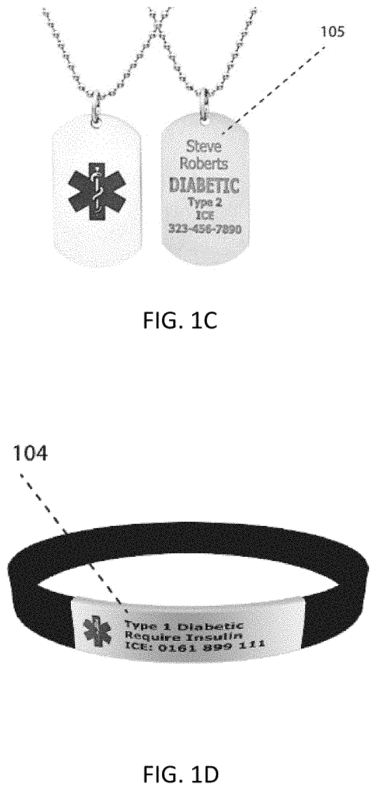

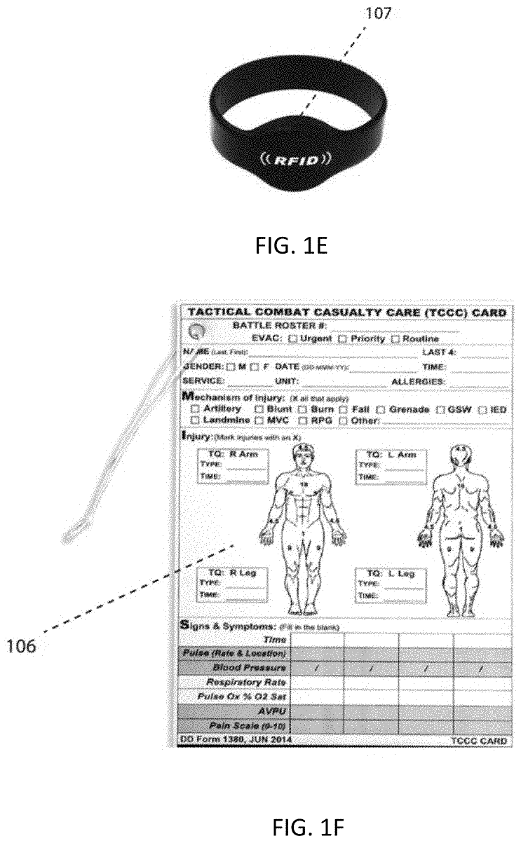

FIGS. 1A-1F illustrate currently used wearable devices to communicate medical information for a patient 100. FIGS. 1A and 1C illustrate example medical necklaces, FIGS. 1B, 1D, and 1E illustrate example medical bracelets, and FIG. 1F illustrates an example medical care card. In many circumstances, a patient 100 will wear a medical wrist band 101 around their wrist 102 as shown in FIG. 1B or a chain with a dog tag style medical ID 103 around their neck as shown in FIGS. 1A and 1C.

In prior devices, a wristband 101 (shown in FIGS. 1B and 1D) typically contains enough information 104 to alert a caregiver to critical care information, but rarely has enough surface area to record all of necessary information for a caregiver. Sometimes the information 104 shown on the device will contain lookup information or identification but this requires the caregiver to have access to an ID server, which may or may not be reachable. Dog tags 105 (shown in FIG. 1C), similar to the bracelets shown in FIGS. 1B and 1D, are limited in area and require Internet access to obtain relevant medical and contact information.

In a similar vein, RFID (radio frequency ID) bracelets 107 (shown in FIG. 1E) can contain additional information which is extracted by placing the band in the field of an RFID reader. While these bracelets can contain more information than can be printed directly on a bracelet or necklace, there are several detracting features of these devices, including the potential for the band to be lost, the need for an RFID reading device to obtain the information stored, and the need for an RFID device to change any of the information on the wrist band.

Another known device for temporarily communicating information about a patient is a placard or card 106 (shown in FIG. 1F) that can be attached to a person or animal. These cards 106 can contain any information the user desires and may sometimes include multiples, each with custom information. While these cards 106 can be customized to provide a large amount of information, they can easily be lost, can be cumbersome to carry, are not prone to modifications, and portray potentially sensitive information in an open environment. The presently disclosed markings and related methods, described below in detail, addresses these shortfalls of the prior art.

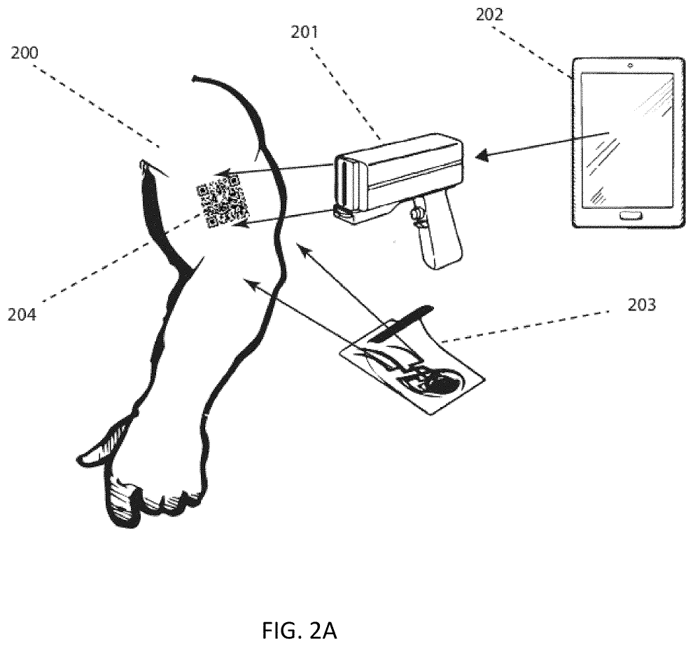

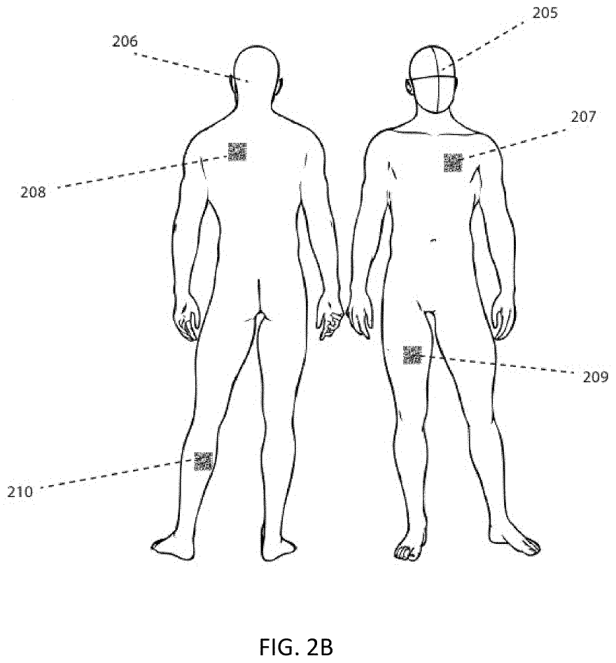

FIGS. 2A and 2B illustrate exemplary techniques and componentry that may be used to apply the disclosed temporary markings to a patient. As shown in FIG. 2A, a computing system 202 may be used to create an encoded marking 204 which is transferred to the body of a person 200 via the use of a handheld printer 201. In some embodiments, the printer 201 may imprint one or more markings 204 on the body of the person (or animal) in any location desired.

Similarly, the marking can be printed on to a transfer media 203 which can then be used to make the marking 204 on the person's body 200, as shown in FIG. 2A. The location of the marking(s) can be anywhere on the body of the person 200, either front 205 or back 206, as shown in FIG. 2B. Markings can be placed on limbs 210 209 or on the torso back 208 or torso front 207. The user may choose to place the marking in an inherently visible 207 place or hide the marking 209, depending if additional privacy is desired.

In some embodiments, the user may also choose to affix several markings, defining the order in which the markings are read or denoting the order of the marking in the encoding itself. The user may also wish to use both direct 201 and indirect 203 marking methods in combination, in some embodiments.

The information originally used to encode the marking 204 can be kept on the computing device 202 or transferred to other systems that will be used to decode the marking 204 in other locations. The decoding of the marking can be done with any suitable code reading device, mobile phone, or computing system.

Markings 204 can appear in any suitable manner. For example, in some embodiments, marking 204 are visible, invisible or a combination thereof. In some embodiments, a single individual may be marked with one or more markings 204 to convey information. In embodiments in which more than one marking 204 is affixed to the individual, the marking can be chained together to capture additional information, overprinted to change data values over time, and/or combined using visible and non-visible marking material. Numerous configurations and variations are possible and contemplated herein.

In some embodiments, markings 204 may include encrypted data, clear text, commands, such as URL locations, and/or application specific data and data masks. The presently disclosed temporary informational markings 204 can, in some embodiments, be used to communicate medical information such as Tactical Combat Casualty care data, after-care instructions, emergency medical information, and/or event access/security data. It should be understood that the terms “temporary informational marking,” “marking 204,” “marking,” and similar variants thereof are used interchangeably herein.

Marking 204 can be implemented with an opaque ink, in some embodiments. In embodiments in which a patient with a lighter skin tone is to be marked, a black or other dark-colored ink may be used for marking 204, whereas in embodiments in which a patient with a darker skin tone is to be marked, a white or other light-colored ink may be used for marking 204. In these and other embodiments, marking 204 may include ink enhancers, such as UV-activated inks, for example. In select embodiments, marking may include a photochromatic ink that includes a UV-activated component such that the ink changes color upon exposure to UV radiation.

If the marking 204 is encoded, the reader will require software which will facilitate the decoding and interpretation of the encoded marking 204. If the marking consists of readable text, the decoding device can simply ascertain the text format (ex: ASCII, UTF etc.) and display the resulting information. The encoding could also consist of pictorial or audible information, which would require the reader to know the format or obtain the format during decoding.

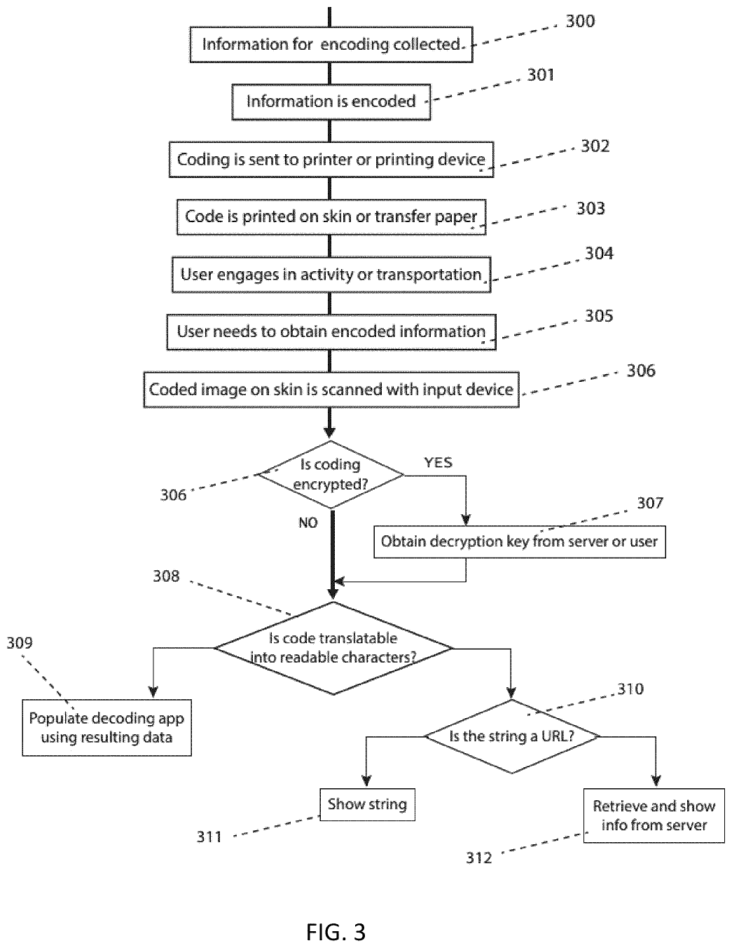

FIG. 3 illustrates an example flow diagram of an exemplary process flow for encoding and decoding a temporary marking. As shown in FIG. 3, initially a user collects the information 300 they wish to communicate via the temporary marking. This information can sometimes, but not always, be gathered using an electronic device that can take the information and encode 301 it into a format that will conform with the marking type to be affixed to the body of the wearer.

Once the coding of the information is complete, the instructions for printing the marking 302 are sent to a printer for printing. These instructions are particular to the specific printer being used as they are normally hardware dependent.

Once the printer is ready to print, the printer can print the marking 303 on the wearer or on a transfer media that is then affixed to the wearer. At the end of this step, the wearer should have a temporary marking on their body that encodes the information desired and can now go about their activities 304.

At some point the user or a third party may want to acquire the information encoded 305 in the temporary marking. The coded marking can then be scanned 206 with an image system which will examine the information contained in the marking.

The decoding software can then examine the contents of the marking to determine if the contents are encrypted 306 or in clear text. This can be accomplished by either apriori knowledge of the encryption requirement or by performing natural language processing on the marking contents.

If the contents are encrypted, the decoding software can obtain a decryption key 307 from either another computing system or the user. This decryption key may, in some embodiments, allow the software to obtain the pure state of the marking contents. If encryption is not found, then the software can proceed to the next step in the decoding process.

Once the decoded data is obtained, 308, the software can determine what information is contained therein 308. The data might pertain to a known data format or mask that allows the decoding software to populate itself or other data sources with the encoded data, or the information might be in a readable 310 format.

If the data is in a readable character format 310, the decoding software may either display the text to the user 311 or take action based upon the command encoded into the text 312 such as displaying the webpage at a URL location.

### EXAMPLES OF USE

There are several ways in which the disclosed methods and devices can be used to improve communication and the transfer of knowledge for a temporary period.

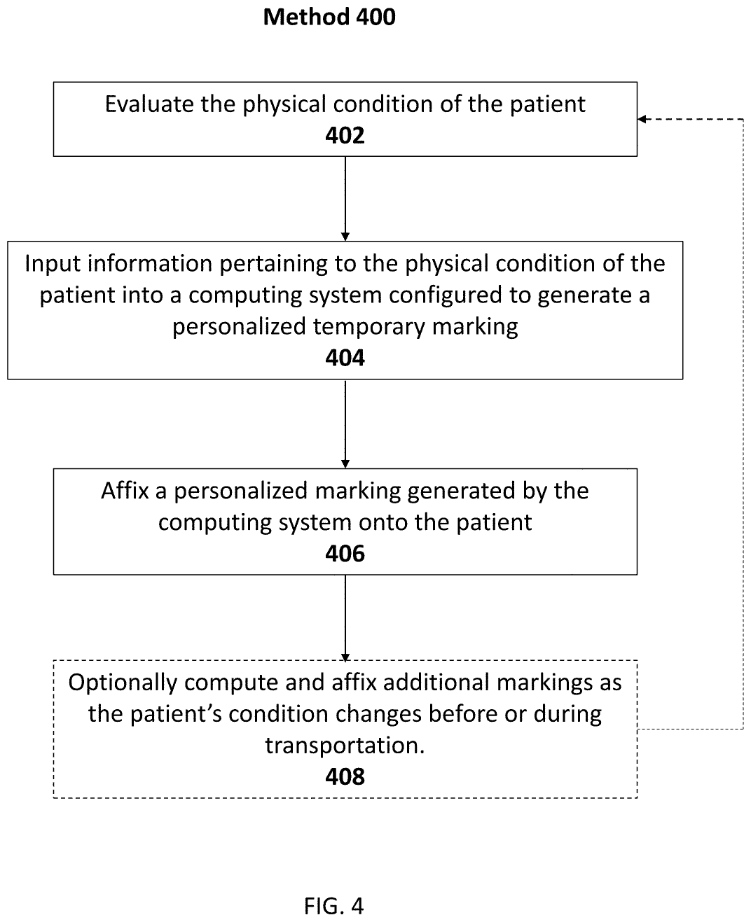

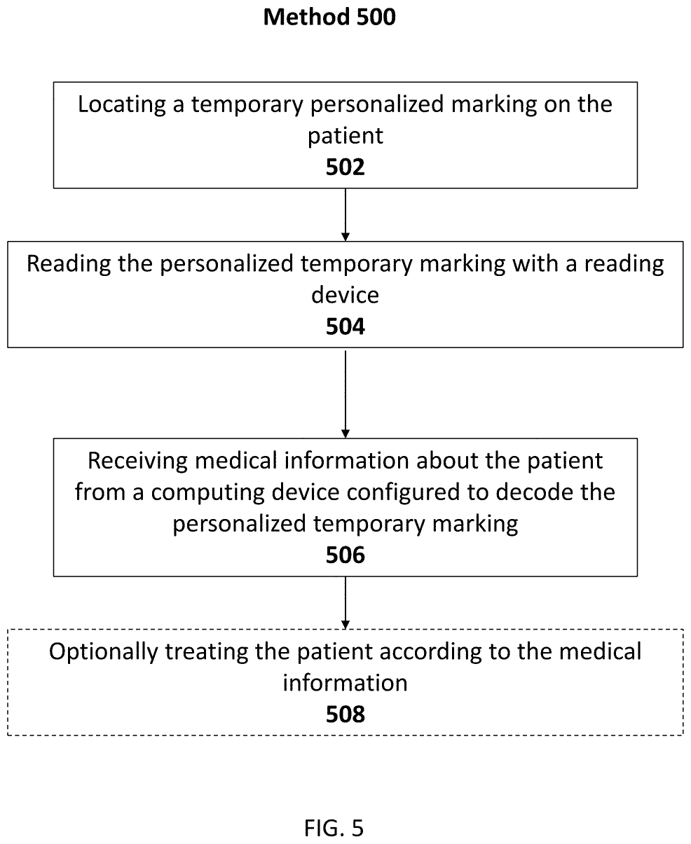

As previously discussed, the present disclosure involves new methods of temporarily marking an individual (alternatively referred to as a ‘patient’) with medical information as well as methods of deciphering and using the medical information marked on the individual to perform medical treatment. FIG. 4 illustrates an example method of temporarily marking an individual and FIG. 5 illustrates an example method of reading the temporary marking and administering medical treatment to the individual. Specifics of the example methods shown in FIGS. 4 and 5 are discussed in detail below.

FIG. 4 illustrates an example method 400 of temporarily marking an individual. As shown in FIG. 4, method 400 includes evaluating the physical condition of the patient (Block 402). In some circumstances, evaluating the physical condition of the patient can include a medical evaluation, physical evaluation, or other type of assessment. The patient can be evaluated by a medical professional or a layperson, in some embodiments. Vital signs and/or other medical diagnostics of the patient can be measured during the evaluation, if appropriate.

Method 400 continues with inputting information pertaining to the physical condition of the patient into a computing system configured to generate a personalized temporary marking (Block 404). The information that is input into the computing system can be numerical in nature and may, in some embodiments, include text, photos, videos, symbols, voice recordings, and/or other types of data. The information can be input into the computing system in any desired manner, such as with keystrokes, audio input, file uploads, and/or by other techniques. In select embodiments, the computing system may provide prompts to facilitate information input. For example, in some embodiments, the computing system may prompt the user for the patient name, then its age, measured vital signs, notes, photos, and/or other relevant information.

Method 400 continues with affixing a personalized temporary marking generated by the computing system onto the patient (Block 406). In some embodiments, the personalized temporary marking may be implemented with temporary printable ink and may be affixed to the patient by a transfer method after the personalized temporary marking is printed. In some embodiments, the personalized temporary marking is fully or partially encoded, making it difficult or impossible to interpret without a decoder.

Method 400 optionally continues with affixing one or more additional markings to the patient. Additional marking(s), if desired, can be affixed to the patient at the same time or subsequent to application of the first marking. Additional markings can be affixed to the patient, in some embodiments, to reflect evolution of the patient's condition over time. Marking a patient with at least two marking can provide the ability to augment and/or enhance data encoded in the original marking. In such embodiments, the resulting data represent the patient's current status as well as the patient's progress over time.

FIG. 5 illustrates an example method 500 of reading a temporary marking on a patient and administering medical treatment to the patient. As shown in FIG. 5, method 500 includes locating a temporary personalized marking on a patient (Block 502). The temporary personalized marking can be located visually, in some embodiments, whereas in other embodiments, the temporary personalized marking can be located by scanning the patient's body with a scanning device. Example scanning devices include but are not limited to cell phones, tablets, personal computers, cameras, and/or laser scanners.

Method 500 continues with reading the personalized temporary marking with a reading device (Block 504). In some embodiments, the reading device may be a scanner, such as a QR scanner or a barcode scanner. The reading device may be in communication with a computing device configured to decode the personalized temporary marking, in some embodiments.

Method 500 continues with receiving medical information about the patient from a computing device configured to decode the personalized temporary marking (Block 506). The medical information may be presented in any desired format. For example, in some embodiments, the medical information may be displayed on an electronic screen or may be printed. The medical information may appear as a medical chart, with diagrams and/or text, as appropriate.

Method 500 optionally continues with treating the patient based on the medical information about the patient generated by the computing device (Block 508). In some embodiments, treating the patient may include administering medication, performing one or more procedures, and/or providing other care to the patient.

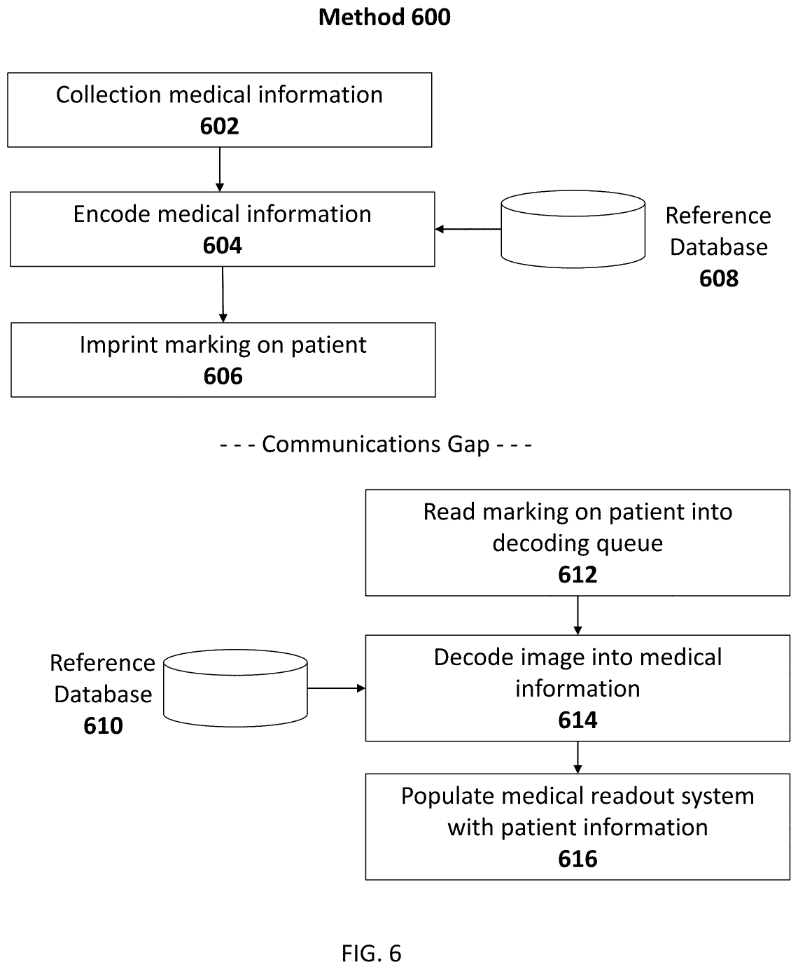

FIG. 6 illustrates an example method 600 for the transmission of data across a communication gap, specific to an example in which medical information is being transmitted. As shown in FIG. 6, method 600 begins with gathering or collecting medical information (Block 602). In some embodiments, a medic can gather medical information from a patient, such as various injuries sustained by the patient and treatments provided to stabilize and address any issues the patient has. Once the medical information has been collected, it can be input into a computing system.

After the medical information has been input into a computing system, method 600 continues with encoding the medical information (Block 604) in order to format the data in a manner that support the compression and expression necessary to create a marking. Encoding the medical information can be accomplished by any suitable technique. For example, in some embodiments, the medical information may be encoded according to a reference database 608. Encoding enhances the compression capability, since the encoding method can take advantage of certain predefined compression techniques. Once the encoding is complete then the marking can be created and imprinted on the patient (Block 606).

In some embodiments, the patient is then transported across one or more communication gaps and put in contact with a remote caregiver. At that point, the remote caregiver can read the marking on the patient (Block 612) using whichever type of device is preferred and adding the marking to a decoding queue.

As shown in method 600, the marking will then be decoded (Block 614) using a decompression algorithm supported by a reference database 610. In some embodiments, reference database will contain the same information as reference database 608 but may not be in communication with reference database 608. Such a configuration can advantageously allow for information to be communicated to distinct locations without direct communication between the databases, to minimize hacking, data overloading, and/or privacy breaches. After decoding is complete, the data that was encoded (pursuant to Block 604) will be revealed. This data will then be populated into a medical reference system (Block 616) used by the remote caregiver to continue the care of the patient. This information can also be persisted to the electronic medical record system, if desired.

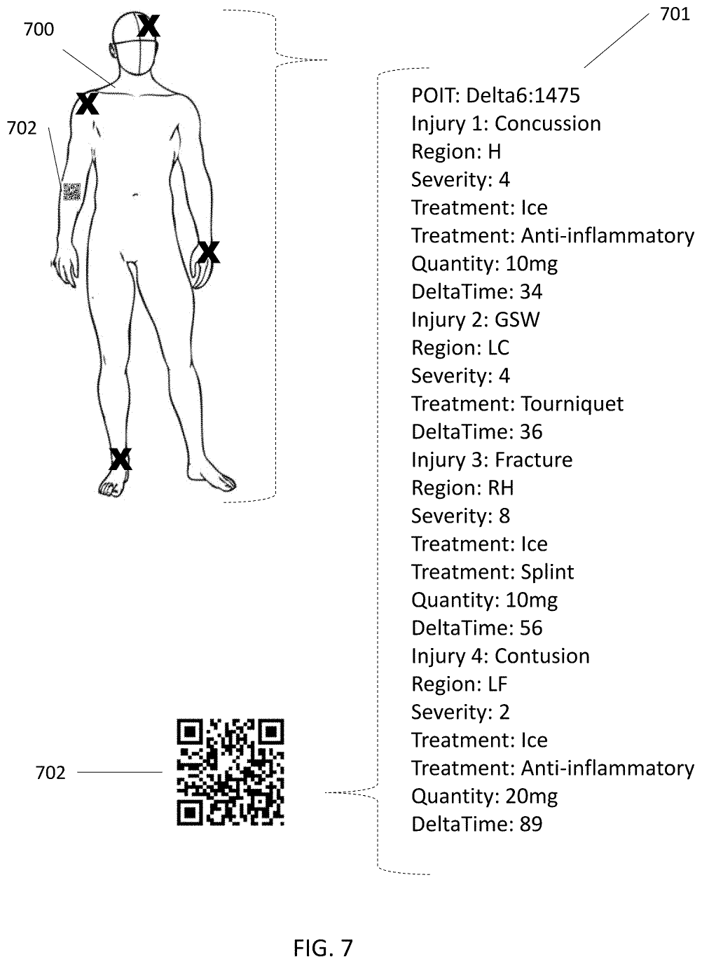

FIG. 7 illustrates exemplary types of injuries and related data that can be encoded into the temporary marking described herein. As shown in FIG. 7, a UI element 700 with various regions marked may be used to depict injuries and locations thereof. In some embodiments, a medic will treat the patient's injuries and record, via the medic's chosen method of collection, the various injuries and applicable treatment. This information 701 can be stored in a computing system used by the medic.

The data contained in this medical information 701 can be encoded into a marking 702 that can be affixed to the patient in a location, such as shown in FIG. 7. When the patient is transported across one or more communicate gaps, the marking 702 can be scanned and the medical information retrieved by personnel present at that location.

In some example embodiments, the disclosed methods and technique are used to encode medical information captured at the Point of Injury (POI) by a medic treating a warfighter. In some such embodiments, TC3 card information can be encoded into a QR code, affixed to the soldier using a temporary tattoo printer, and then read by the care team at the triage center to which the warfighter was medically evacuated. In some such embodiments, all treatment information for the warfighter is present the moment the patient enters triage as well as during the evacuation process. Evacuation care givers could also augment the existing tattoo by adding additional tattoos to capture additional medical care information pertaining to treatment provided while in transport.

Post-care documentation is also a very viable application for temporary markings. Often when a patient leaves a healthcare facility, they are provided with medical information, usually on paper. These paper instructions are often lost or are only visible via a web portal that the patient can access. If other caregivers need to reference these instructions it can be problematic or, over time, the instructions can be lost. By encoding treatment documentation onto the patient with a temporary marking or tattoo, which then points to a secure or encrypted URL, anyone can scan the marking with a smartphone or tablet and retrieve treatment instructions as needed. This eliminates the printing, shipping, and potential loss of paperwork involved in post-care treatment situations.

Additionally, emergency travel and medical information is often subject to change and the information can be too voluminous to be communicated via a text readable wearable. In addition, a wearable can be misplaced or stolen. By temporarily marking a traveler with a QR code (or similar encoding), like the out-patient use case, anyone who is providing care can scan the marking or code with a mobile phone and retrieve the healthcare information required to properly care for the patient. This is especially important if the patient is not conscious. Temporary markings of this nature also enable changes to be easily made to the relevant medical information. For example, a medical bracelet cannot be modified without purchasing a new one, but a temporary marking or tattoo can be encoded with new medical information easily and as often as necessary.

Entertainment and event access can also be tracked using temporary markings. For example, it is common for patrons at an event or bar to be affixed with an ink stamp, however this stamp could be further encoded with age, entertainment access, billing, and security information, using the disclosed techniques. This would allow event management to control access and purchases by simply scanning the individual's marking(s).

The foregoing description of the embodiments has been presented for the purposes of illustration and description. It is not intended to be exhaustive or to limit the disclosure to the precise form disclosed. Many modifications and variations are possible in light of this disclosure. It is intended that the scope of the present disclosure be limited not by this detailed description, but rather by the claims appended hereto.

What is claimed is:

1. A method of temporarily marking a patient with medical information, the method comprising:
evaluating a physical condition of the patient;
inputting information pertaining to the physical condition of the patient into a computing system configured to generate a personalized temporary marking; and
affixing the personalized temporary marking to the patient generated by the computing system.

2. The method of claim 1, wherein evaluating the physical condition of the patient includes a medical evaluation performed by a medical professional.

3. The method of claim 1, wherein the information pertaining to the physical condition of the patient includes text, numbers, audio, video, and/or photos.

4. The method of claim 1, wherein the personalized temporary marking is partially or fully encoded.

5. The method of claim 1, wherein the patient is a human or an animal.

6. The method of claim 1, wherein the personalized temporary marking is imprinted directly onto the patient's skin in the form of a temporary tattoo.

7. The method of claim 1 further comprising affixing one or more additional personalized temporary markings to the patient generated by the computing system.

8. The method of claim 1, wherein the personalized temporary marking is implemented with an ink comprising a UV-activated component.

9. A method of reading a temporary marking on a patient, the method comprising:
locating a personalized temporary marking on the patient;
reading the personalized temporary marking with a reading device; and
receiving medical information about the patient from a computing device configured to decode the personalized temporary marking.

10. The method of claim 9 further comprising administering one or more medical treatments to the patient based on the medical information decoded by the computing device.

11. A method of transmitting medical data across a communication gap, the method comprising:
at a first location:
collecting medical information regarding a patient;
inputting the medical information into a computing system in communication with a first reference database, wherein the computing system is configured to encode the medical information using the first reference database;
receiving an encoded marking from the computing system; and
marking the patient with the encoded marking,
transporting the patient from the first location across a communications gap to a second location; and
at a second location:
reading the encoded marking on the patient using a scanning device and providing the reading to a decoding queue, wherein the decoding queue is in communication with a second reference database;
receiving decoded medical information regarding the patient from the decoding queue; and
treating the patient according to the decoded medical information.

12. The method of claim 11, wherein the first reference database and the second reference database are not in communication.
